(19) United States
(12) Patent Application Publication
(10) Pub. No.: US 2013/0095188 A1
(43) Pub. Date: Apr. 18, 2013

(54) Method of Encapsulating a Material Using Solvent Removal Technique, and Microspheres/Nanospheres/Matrix Made Therefrom Having Sustained Release Properties

(71) Applicants: Edith Mathiowitz, Brookline, MA (US); Daniel Y. Cho, Pawtucket, RI (US); Roshni Rainbow, Quincy, MA (US)

(72) Inventors: Edith Mathiowitz, Brookline, MA (US); Daniel Y. Cho, Pawtucket, RI (US); Roshni Rainbow, Quincy, MA (US)

(21) Appl. No.: 13/624,941

(22) Filed: Sep. 23, 2012

Related U.S. Application Data
(60) Provisional application No. 61/627,569, filed on Oct. 14, 2011.

(51) Int. Cl. A61J 3/07; A61K 31/65; A61P 31/04; A61K 9/50

(52) U.S. Cl. 424/497; 514/152; 264/4.1

(57) ABSTRACT

A method of encapsulating a material includes providing a polymer solution including a solvent, and an aqueous solution including a hydrophilic material, mixing the polymer and aqueous solutions, sonicating the mixed solution to obtain a water-in-oil (W/O) emulsion, mixing the water-in-oil emulsion with an oil solution, sonicating the mixed solution to obtain a water-in-oil-in-oil (W/O/O) emulsion, and stirring the water-in-oil-in-oil emulsion in a bath to form a precipitate of encapsulated material and separate the solvent.

### CROSS-REFERENCE TO RELATED APPLICATIONS

The present application claims priority on prior U.S. Provisional Application Ser. No. 61/627,569, filed Oct. 14, 2011, which is hereby incorporated herein in its entirety by reference.

### STATEMENT REGARDING FEDERALLY SPONSORED RESEARCH OR DEVELOPMENT

This work was partially supported by the Office of Academic Affiliations, Department of Veterans Affairs. The U.S. Government therefore has certain rights in the invention.

### FIELD AND BACKGROUND OF THE INVENTION

The present invention is generally directed to microencapsulation techniques, and more particularly to encapsulating a material using a solvent removal technique, and microspheres/nanospheres/matrix made therefrom having sustained release properties.

Solvent removal is a modification of organic phase separation originally developed as an alternative to solvent evaporation for the encapsulation of therapeutics within polyanhydrides due to the extreme lability of the anhydride bond in the presence of water to preserve chemical integrity by replacing the aqueous bath with an oil phase (Mathiowitz et al. 1988). With advancements in the field of microencapsulation, many similar techniques have been developed resulting in the similar nomenclature, which includes terms such as solvent extraction and solvent diffusion.

Traditionally, solvent removal relies on the oil-in-oil (O/O) emulsification of a polymer solution into a continuous phase, with both organic systems having partial solubility in each other. During sphere formation, the extraction of the organic solvent into this continuous phase causes a decrease in the local solubility of the polymer in the polymer-rich droplets resulting in particle formation, a more rapid process compared to many other encapsulation techniques including solvent evaporation (Ciombor et al. 2006).

Despite the potential advantages of this method, very few groups have evaluated solvent removal as a means of encapsulating hydrophilic small molecule drugs and research has instead been focused on the encapsulation of solid proteins with this method. Studies have confirmed the best correlations between theoretical and actual loadings were achieved when encapsulating hydrophilic proteins by solvent removal because the microencapsulation process occurs in a hydrophobic environment (Mathiowitz et al. 1990; Mathiowitz et al. 1988). Our group has previously established the use of this method for the encapsulation of proteins within biodegradable polyesters, specifically poly(lactic-co-glycolic acid) (PLGA) (Ciombor et al. 2006).

DOX is a synthetic tetracycline-derivative antibiotic, commonly used to treat bacterial infections of the skin (Smith et al. 2005) and eye (Maatta et al. 2006; Quarterman et al. 1997; Stewart et al. 2005), as well as infections associated with malaria (Dahl et al. 2006; Lalloo et al. 2007), Lyme's disease (Zeidner et al. 2004), and gingivitis (Mundargi et al. 2007), among others. The encapsulation of DOX in PLGA has been described previously using solvent evaporation or double emulsion techniques, most commonly using water/oil/water (W/O/W) (Mundargi et al. 2007; Patel et al. 2008). In such methods, encapsulation efficiencies remained no higher than 25% due to loss of DOX to the secondary aqueous phase during fabrication. Despite efforts to circumvent this loss through the use of a W/O/O double emulsion with solvent evaporation for DOX encapsulation in PLGA, encapsulation efficiencies of no greater than 26% have been reported (Feng et al. 2010).

While protein encapsulation has been demonstrated using solvent removal as a method to minimize the loss of water-soluble proteins during encapsulation, as of this time, to our knowledge, this method has not been explored for the encapsulation of hydrophilic drug molecules. Here, in this work, we describe the encapsulation of a model hydrophilic drug, doxycycline (DOX), in PLGA using a W/O/O double emulsion solvent removal technique to minimize DOX loss during the encapsulation process. In addition, we have characterized the systems for encapsulation efficiencies, particle size, particle surface morphology, and DOX release kinetics. Additional analysis includes thermal, spectral and x-ray diffraction.

### ASPECTS OF THE INVENTION

The present disclosure is directed to various aspects of the present invention.

One aspect of the present invention is to encapsulate a hydrophilic compound or molecule using a solvent removal technique.

Another aspect of the present invention is to encapsulate a hydrophilic compound or molecule using a water-in-oil-in-oil (W/O/O) solvent removal technique.

Another aspect of the present invention is to encapsulate a low molecular weight hydrophilic compound or molecule using a solvent removal technique. The compound or molecule is preferably a drug, such as a tetracycline or a derivative thereof. More specifically, the drug is doxycycline (DOX).

Another aspect of the present invention is to prepare a polymer matrix including a hydrophilic compound or molecule using a solvent removal technique.

Another aspect of the present invention is to prepare a polymer matrix including a hydrophilic compound or molecule using a water-in-oil-in-oil (W/O/O) solvent removal technique.

Another aspect of the present invention is to prepare a polymer matrix including a low molecular weight hydrophilic compound or molecule using a solvent removal technique. The compound or molecule is preferably a drug, such as a tetracycline or a derivative thereof. More specifically, the drug is doxycycline (DOX).

Another aspect of the present invention is to prepare microparticles, such as microspheres and/or nanospheres, including a hydrophilic compound or molecule using a solvent removal technique.

Another aspect of the present invention is to prepare microparticles, such as microspheres and/or nanospheres, including a hydrophilic compound or molecule using a water-in-oil-in-oil (W/O/O) solvent removal technique.

Another aspect of the present invention is to prepare microparticles, such as micropsheres and/or nanospheres, including a low molecular weight hydrophilic compound or molecule using a solvent removal technique. The compound or molecule is preferably a drug, such as tetracycline or a derivative thereof. More specifically, the drug is doxycycline (DOX).

Another aspect of the present invention is to provide an encapsulated material, such as microspheres and/or nanospheres, wherein the loading of a hydrophilic compound or molecule is upto 20% by weight. The compound or molecule preferably has a low molecular weight and includes a drug, such as a tetracycline or a derivative thereof. More specifically, the drug is doxycycline (DOX).

Another aspect of the present invention is to provide an encapsulated material, such as microspheres and/or nanospheres, wherein a hydrophilic compound or molecule is dispersed in an amorphous state. The compound or molecule is preferably a drug such as a tetracycline or a derivative thereof. More specifically, the drug is doxycycline (DOX).

Another aspect of the present invention is to provide an encapsulated material, such as microspheres and/or nanospheres, including a hydrophilic compound or molecule. The encapsulated material has the capacity to sustain release of the hydrophilic compound or molecule for upto 85 days or more.

Another aspect of the present invention is to provide an encapsulated material, such as microspheres and/or nanospheres, having an average particle diameter of about 600 nm to about 19 μm, and an encapsulated efficiency ranging from about 46% to about 83%.

Another aspect of the present invention includes a method of encapsulating a material, including providing a polymer solution including a solvent, and an aqueous solution including a hydrophilic material, mixing the polymer and aqueous solutions, sonicating the mixed solution to obtain a water-in-oil (W/O) emulsion, mixing the water-in-oil emulsion with an oil solution, sonicating the mixed solution to obtain a water-in-oil-in-oil (W/O/O) emulsion, and stirring the water-in-oil-in-oil emulsion in a bath to form a precipitate of encapsulated material, and separate the solvent.

Another aspect of the present invention includes a method of incorporating a material in a polymer matrix, including providing a polymer solution including a solvent, and an aqueous solution including a hydrophilic material, mixing the polymer and aqueous solutions, sonicating the mixed solution to obtain a water-in-oil emulsion, mixing the water-in-oil emulsion with an oil solution, sonicating the mixed solution to obtain a water-in-oil-in-oil emulsion, and stirring the water-in-oil-in-oil emulsion in a bath to form a precipitate of a polymer matrix including the hydrophilic material, and separate the solvent.

Another aspect of the present invention includes a method of preparing pharmaceutical particles, including providing a polymer solution including a solvent, and an aqueous solution including a hydrophilic material, mixing the polymer and aqueous solutions, sonicating the mixed solution to obtain a water-in-oil emulsion, mixing the water-in-oil emulsion with an oil solution, sonicating the mixed solution to obtain a water-in-oil-in-oil emulsion, stirring the water-in-oil-in-oil emulsion in a bath to form a precipitate of particles, and separate the solvent, removing and flash freezing the particles, and lyophilizing the frozen particles for a predetermined time period.

Another aspect of the present invention includes an encapsulated material, which includes a polymer matrix having a low molecular weight hydrophilic compound or molecule dispersed in an amorphous state. The polymer matrix includes poly(lactic-co-glycolic acid) (PLGA) having a glass transition temperature (T_{G}) of from about 34° to about 39°.

Another aspect of the present invention includes a microsphere or nanosphere, which includes a polymer sphere comprised of poly(lactic-co-glycolic acid). The polymer sphere includes upto 20% by weight of an antibiotic or a drug, and has a capacity of upto 85 days or more for release of the antibiotic or drug.

In summary, the present invention is based, at least in part, on a study reporting on the development of a modified solvent removal method for the encapsulation of hydrophilic drugs within poly(lactic-co-glycolic acid) (PLGA). Using a water/oil/oil (W/O/O) double emulsion, hydrophilic doxycycline (DOX) was encapsulated within PLGA spheres with particle diameters ranging from approximately 600 nm to 19 μm. Encapsulation efficiencies of up to 83% can be achieved for theoretical loadings ranging from 1-20% (w/w). DOX displayed biphasic release of upto 85 days or more, with nearly complete release at the end of this time course. 1% salt was added to the formulations to examine its effects on DOX release, and it was reported that salt modulated release only by increasing the magnitude of initial drug release without altering release kinetics. FTIR analysis indicated no characteristic differences between DOX-loaded and unloaded PLGA spheres. DSC analysis revealed the absence of a first-order transition in the range of the DOX melting temperature, and together with x-ray diffraction analysis, suggests that there may be a molecular dispersion of the DOX within the spheres and that the encapsulated DOX may be in an amorphous state, which could explain the slow, prolonged release of the encapsulated hydrophilic drug.

### BRIEF DESCRIPTION OF THE DRAWINGS

One of the above and other aspects, novel features and advantages of the present invention will become apparent from the following detailed description of the non-limiting preferred embodiment(s) of invention, illustrated in the accompanying drawings, wherein:

FIG. 1 illustrates low and high magnification SEM micrographs of PLGA particles loaded with 10% DOX (Images A and E), 5% DOX (Images B and F), 1% DOX (Images C and G), and 0% DOX (Images D and H);

FIG. 2 illustrates low and high magnification SEM micrographs of PLGA particles loaded with 10% DOX and 1% NaCl (Images A and E), 5% DOX and 1% NaCl (Images B and F), 1% DOX and 1% NaCl (Images C and G), and 1% NaCl only (Images D and H);

FIG. 3 illustrates cumulative fractional release of DOX from loaded PLGA spheres: (A) formulations without salt and (B) formulations loaded with 1% NaCl. Inset graphs show first 24 hours of release study;

FIG. 4 illustrates FTIR spectra for (a) DOX, (b) control PLGA spheres, (c) 1% DOX PLGA spheres, and (d) 1% DOX and 1% NaCl PLGA spheres; and

FIG. 5 illustrates XRD diffractograms of DOX, PLGA 50:50, 1% and 20% DOX loaded spheres, and unloaded control spheres.

### DETAILED DESCRIPTION OF THE PREFERRED EMBODIMENT(S) OF THE INVENTION

### Materials and Method

### Materials

Poly(lactic-co-glycolic acid) (PLGA) 50:50 (Resomer RG502; I.V=0.16−0.24 dL/g in CHCl₃) was purchased from Boehringer Ingelheim (Germany). Doxycycline hydrochloride (DOX) was acquired from Clontech, Inc. (Mountain View, Calif.). Sodium chloride (NaCl) and sodium hydroxide (NaOH) were both obtained from Sigma Aldrich (St. Louis, Mo.). Silicon oil (100 CST) was obtained from Dow Corning (Midland, Mich.). All solvents were purchased from Fisher Scientific (Hampton, N.H.) and were of the highest commercial grade available. For release studies, phosphate buffered solution (PBS, pH=7.4) was obtained from Invitrogen (Carlsbad, Calif.).

### Fabrication of Micro- and Nanospheres

PLGA micro- and nanospheres were fabricated using a water/oil/oil (W/O/O) double emulsion solvent removal process adapted and modified from the method described by Ciombor et al (Ciombor et al. 2006). For all formulations, 500 mg of PLGA was dissolved in 15 mL of dichloromethane (DCM), while the appropriate amount of DOX equivalent to the percentage dry weight of drug to dry weight of the polymer was dissolved in 300 μL of distilled water. The DOX solution was added to the polymer solution and probe sonicated using an Ultrasonic Homogenizer CV26 (Cole-Farmer, Vernon Hills, Ill.) at 40% amplitude for one minute to create the primary W/O emulsion. This emulsion was then poured into 80 mL of a 20% v/v DCM/silicon oil solution and probe sonicated for an additional 2 minutes to create the secondary W/O/O emulsion. The resulting double emulsion was poured into a 1 L bath of petroleum ether and stirred at 2000 RPM for 5 minutes to allow for solvent removal and precipitation of the polymer to form nanospheres. The nanospheres were collected using a positive pressure filtration column with a 0.2 μm PTFE filter (Millipore, Billerica, Mass.) and washed with petroleum ether to remove the silicon oil. The final product was flash frozen, lyophilized for 2 days, and stored at −20° C.

DOX loading varied from 1%-20% w/w (drug/polymer). For formulations containing 1% NaCl, 5 mg of NaCl was dissolved in the distilled water solution containing DOX prior to addition to the polymer solution to form the first emulsion. Unloaded nanospheres were also fabricated using the above method and served as a negative control. The yields of all formulations were ˜60%.

### Encapsulation Efficiency

To determine the DOX-loading of the nanospheres, 10 mg of the nanospheres were dissolved in 2 mL of 0.1 N NaOH. To insure complete degradation of the spheres, samples (n=3) were placed on a rotating rack for 24 hours. All samples were dissolved in the dark to prevent any degradation of the drug in light. The resulting solutions were filtered using a 0.2 μm PTFE syringe filter (National Scientific, Rockwood, Tenn.) and analyzed for DOX concentration via UV spectrophotometry at an absorbance of 255 nm using a Spectromax Absorbance plate reader (Molecular Devices, Sunnydale, Calif.) and Softmax Pro software (Molecular Devices, Sunnydale, Calif.). Encapsulation efficiency was defined as the ratio of the actual DOX loading to theoretical loading (×100). All samples were run in triplicate and data is reported as mean±standard deviation.

### Particle Sizing

The volume average and number average particle sizes for all formulations were determined by Coulter laser diffraction particle size analysis. 5-7 mg of each formulation was suspended in an aqueous solution of 1% (w/v) polyvinylpyrrolidone (PVP) and 1% (w/v) sodium lauryl sulfate (SLS) with bath sonication and analyzed using a Beckman-Coulter LS230 Laser Diffraction Particle Size Analyzer (Beckman-Coulter, Brea, Calif.). Samples suspended in 1% PVP and 1% SLS were also analyzed for polydispersity and zeta potential using dynamic light scattering (DLS) with a Malvern Zetasizer Nano (Worcestershire, United Kingdom).

### Scanning Electron Microscopy

Scanning electron microscopy was used to confirm particle size and evaluate the surface morphology of all formulations. Samples were mounted onto a carbon-backed adhesive and sputter-coated with gold-palladium for 6 minutes at 20 mA with an Emitech K550 sputter coater (West Sussex, United Kingdom) and visualized using a Hitachi S-2700 scanning electron microscope (Hitachi, Peoria, Ill.) with an accelerating voltage of 8 kV.

### DOX Release Kinetics

DOX release from particles was quantified over the course of 85 days in vitro. 10 mg of nanospheres (n=6 for each formulation) were suspended in 1.25 mL of PBS and incubated in a 37° C. incubator with 5% CO₂. At every time point (1, 2, 4, and 8 hours; 1, 2, 3, 5, 7, 14, 21, 28, 35, 42, 49, 56, 63, and 85 days), nanospheres were removed from the incubator and centrifuged at 10,000 RPM for 5 minutes. One mL of supernatant was then removed and stored at −20° C. for later analysis. One mL of fresh PBS was then added to the nanospheres. Spheres were re-suspended by vortexing briefly and returned to the incubator until the next time point. Prior to quantifying the DOX concentration in the collected samples using UV spectrophotometry, each solution was filtered using a 0.2 μm PTFE syringe filter to remove any polymeric debris. All samples were protected from light and run in triplicate; data shown as mean±standard deviation. Fractional release at each time point was normalized to the total mass released on the last day of the study.

### FTIR Spectral Studies

Fourier-transform infrared spectroscopy (FTIR) was used to study the chemical interactions between DOX and the polymer. FTIR analysis was performed on each formulation using a Perkin-Elmer Spectrum One FTIR (Waltham, Mass.) and analyzed using Spectrum software (Perkin-Elmer, Waltham, Mass.). Samples were scanned in a range between 4000 and 650 cm⁻¹. All measurements were taken at room temperature.

### Thermal Analysis

Differential scanning calorimetry was used to determine the thermal properties of the polymer, drug and all formulations. Approximately 5 mg of each formulation was sealed into aluminum sample pans and analyzed using a Model DSC7 (Perkin-Elmer, Waltham, Mass.) equipped with controller model TAC7/DX (Perkin-Elmer, Waltham, Mass.). After equilibration at −20° C. for 1 minute, samples were heated to 200° C. then cooled to −20° C. and reheated to 200° C. at a rate of 10° C./min. Thermograms were analyzed using Perkin-Elmer Thermal Analysis software for the calculation of glass transition temperatures (T_{G}), melting temperatures (T_{M}) and changes in enthalpy (ΔH) from the first heat.

### X-ray Powder Diffraction Analysis

The crystallinity of the drug, PLGA 50:50, 1% and 20% loaded microspheres, and unloaded controls was confirmed using an automated X-ray diffractometer (Siemens Diffraktometer D5000, Munich, Germany). The diffraction angles (28) ranged from 6° to 60° with sampling intervals of 0.02° s⁻¹. Diffraction signal intensity was monitored and processed using DiffracPlus Software (Bruker AXS, Vienna, Austria).

### Results and Discussion

### Formulations

A W/O/O double emulsion solvent removal method (Ciombor et al. 2006) modified to successfully fabricate DOX-loaded PLGA micro- and nanospheres. Spheres were fabricated by first dispersing an aqueous solution of DOX (with or without salt) in PLGA dissolved in DCM using probe sonication to create a W/O emulsion. The addition of this primary W/O emulsion to the secondary oil phase consisting of silicon oil and DCM with further sonication resulted in the formation of a stable secondary emulsion. The presence of DCM in the silicon oil phase during the secondary emulsification was used to reduce the rate of diffusion of the solvent from the polymer phase, thereby preventing premature polymer precipitation and producing a stable emulsion consisting of the aqueous droplets of drug distributed within the polymer rich organic phase in a continuous phase of oil. While the use of sonication during the formation of the secondary emulsion would be most likely destructive to proteins, we believe it allows for a finer, stable W/O/O emulsion for small hydrophilic molecules, such as DOX, thereby making this method attractive for the encapsulation of these types of molecules. The addition of this secondary emulsion into excess petroleum ether (a non-solvent for the polymer) resulted in the diffusion of silicon oil and DCM into the non-solvent bath, causing rapid, controlled precipitation of the polymer droplets into discreet spheres. A total of ten formulations were employed for encapsulation of DOX within PLGA microspheres (Table 1).

DOX loadings ranged from 0-10% (w/w), and each formulation was fabricated with and without the addition of 1% salt, which served as an osmotic agent to modulate the release of DOX from the spheres.

With the exception of spheres containing 10% DOX, encapsulation efficiencies for all formulations, unsalted and salted, were above 50% with efficiencies decreasing with respect to increased drug loading as indicated in Table 1. The encapsulation efficiencies achieved in this work were nearly 2-fold higher than those achieved for the encapsulation of DOX within PLGA microspheres using optimized W/O/W double emulsion solvent evaporation methods (Mundargi et al. 2007; Patel et al. 2008). For the encapsulation of hydrophilic drugs, such as DOX, low encapsulation efficiencies associated with such methods are attributed to the significant loss of drug into the aqueous bath from the primary aqueous phase during the solvent evaporation process. By utilizing solvent removal and by replacing the aqueous bath with hydrophobic oils and organic non-solvents, drug loss during particle formation was minimized, resulting in higher encapsulation efficiencies.

Particle size analysis of the microspheres by laser diffraction revealed a volume average diameter of 12.42±10.09 to 18.69±13.48 μm and number average diameters of 676±532 to 846±756 nm across all formulations, regardless of total drug loading as seen in Table 1. Previous attempts at microsphere fabrication using a W/O/O double emulsion solvent removal method by our group produced microspheres in the range of 50-100 μm (Ciombor et al. 2006). By increasing the shear rate of the emulsification steps and optimizing the ratio of solvents and non-solvents, we were able to significantly decrease particle size while encapsulating a small hydrophilic drug with high encapsulation efficiencies. Studies by other group using doxycycline encapsulation in PLGA with a W/O/W double emulsion solvent evaporation process were able to successfully prepare microspheres in the ranges of 27 to 200 μm in diameter with encapsulation efficiencies of up to only 25% resulting in far lower actual drug loadings (Mundargi et al. 2007; Patel et al. 2008). Furthermore, Feng et al recently reported encapsulation efficiencies no higher than 26% for DOX in PLGA using an alternative W/O/O solvent evaporation method that relies on evaporation of the solvent from the secondary emulsion to ultimately form microspheres, but which indicates no improvement over prior solvent evaporation methods (Feng et al. 2010). Using our modified W/O/O solvent removal method, we were able to produce small diameter spheres with significantly higher drug loadings and encapsulation efficiencies.

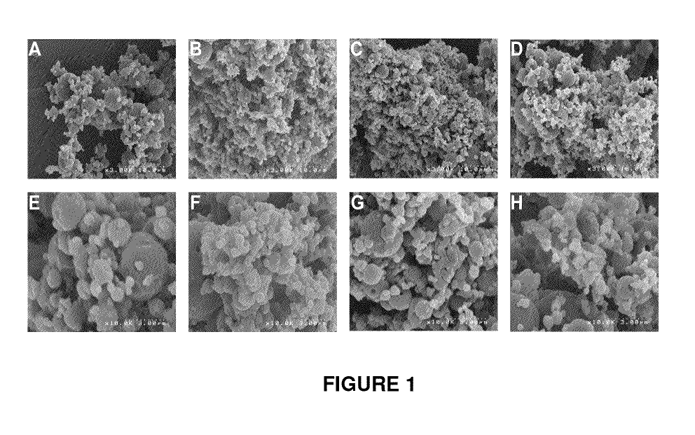

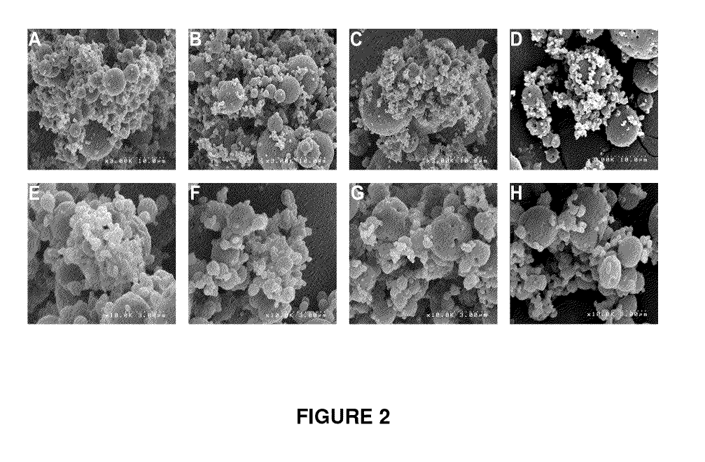

Given the significant difference in the volume average and number average particle sizes, we can conclude that this method generates a population of polydisperse microspheres. We propose two potential explanations for the wider size distribution of the formulations. First, the high shear rates used in the emulsification process combined with the viscosity of the silicon oil during the formation of the secondary W/O/O emulsion may result in the formation of inconsistently sized polymer-rich droplets dispersed within the continuous phase of silicon oil and it is the size of these droplets that determine the final particle size after solvent removal. In addition, it is possible that during the addition of the secondary emulsion to the petroleum ether for solvent removal, the small droplets of the polymer-rich phase may undergo either coalescence or Ostwald ripening due to the destabilization of the emulsion resulting in the formation of larger microspheres. The inclusion of salt in the formulations resulted in slightly smaller particles with narrower size distributions. The polydispersity of the formulations was confirmed by SEM analysis (FIGS. 1 and 2). Furthermore, DLS analysis revealed that the zeta-potential of the microspheres were neutral (data not shown), which suggests a high tendency for aggregation due to hydrophobic interactions

### Morphology

Surface morphology was examined using SEM (FIGS. 1 and 2). For all formulations, a small number of larger, spherical microspheres were observed. A significant number of aggregates consisting of smaller, spherical micro- and nanospheres were also observed. Loading both with respect to DOX and salt did not significantly change the surface morphology. All formulations had an intact outer surface, with small micro- and nano-scale pores observed on some of the larger microspheres.

### DOX Release Kinetics

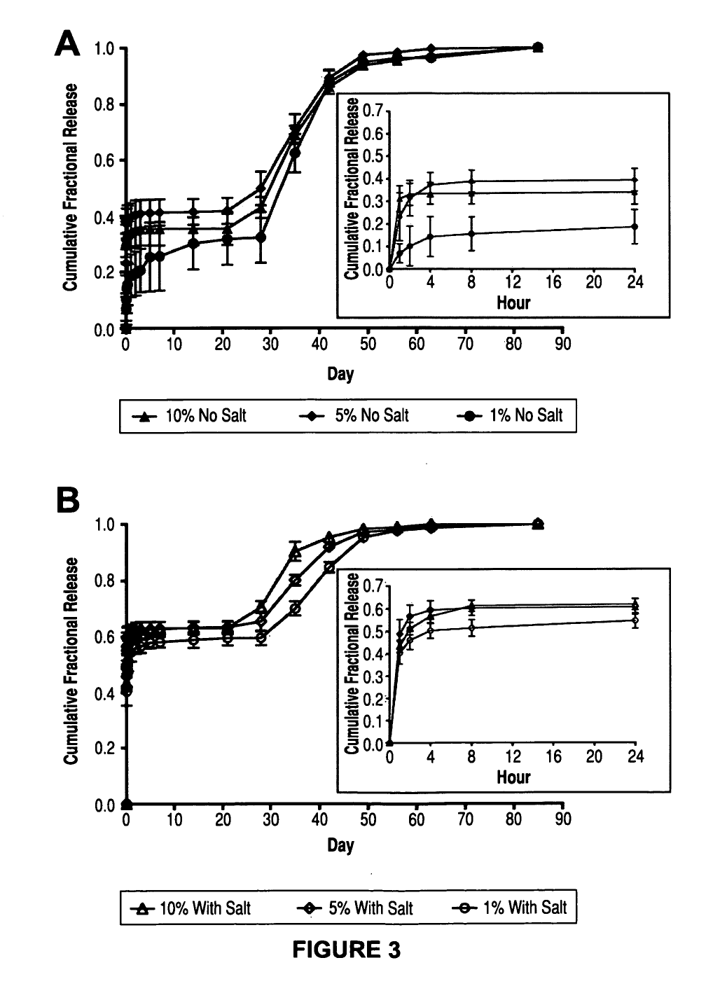

The in vitro DOX release from PLGA microspheres fabricated using the modified solvent removal method is presented in FIG. 3. For all formulations, unsalted or salted, we observed a classic biphasic PLGA release profile with an initial burst due to diffusion of drug molecules trapped on the surface or have access to the surface via pores in the polymeric matrix of the spheres followed by a lag phase and a subsequent, secondary burst due to the bulk degradation of the polymeric matrix core.

Previous studies have reported much shorter durations of release for DOX from PLGA with the majority of the drug being released between 48 hours and 12 days (Mundargi et al. 2007; Patel et al. 2008; Liu et al. 2004) for W/O/W solvent evaporation and only up through 20 days for the W/O/O solvent evaporation (Feng et al. 2010). Rapid release suggests entrapment of the drug on or near the particle surface and which is a result of the method of fabrication. In contrast, the formulations presented in this work allow for sustained drug release to extend out through 85 days. The biphasic release kinetics are suggestive of the control of drug release by both degradation of PLGA (Tan, L. P. et al. 2009) and dispersion of the drug throughout the matrix.

To form emulsions of the aqueous drug in the PLGA organic phase, other methods have utilized vortexing, which may contribute to uneven dispersion of the large aqueous droplets of drug within the polymer matrix and the subsequent rapid drug release. The sustained release of a small, hydrophilic drug from a biodegradable polymeric matrix over the course of nearly three months, as illustrated in FIG. 3, can be explained in part by utilization of sonication during emulsion formation. The resulting formation of very small aqueous droplets of the drug and thorough dispersion of these droplets within the PLGA matrix contributes to the slower, sustained release of DOX, which is predominantly dependent on the degradation of the polymer.

The use of salts as excipients in bioerodible polyester drug delivery systems have been shown to alter release of encapsulated drug by modulating the swelling of the polymeric matrix (Webber et al. 1998; Zhang et al. 1997). The addition of salt into our formulations only affected release by increasing the magnitude of the initial burst of DOX (insets, FIGS. 3A and 3B) without affecting the kinetics or sustainability of release. This is in contrast to the work seen by Webber et al, who showed the ability to modulate the release of tetracycline from PLGA slabs using 1% NaCl to achieve nearly zero-order release kinetics (Webber et al. 1998). This may be the result of the different geometry and size of microspheres and slabs, with spheres exhibiting a higher surface area for drug release relative to slabs.

### DOX-PLGA Interactions Using FTIR Analysis

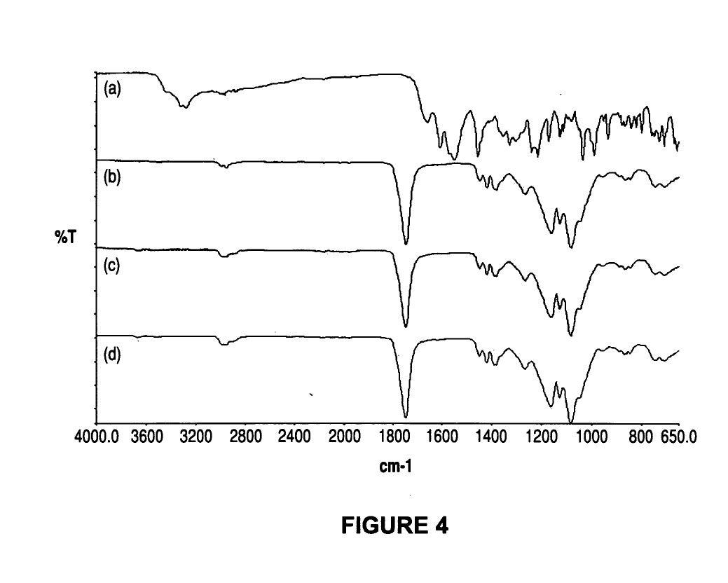

FTIR was used to evaluate the interactions between the PLGA and the encapsulated doxycycline in each formulation; the spectra for bulk DOX, control microspheres, and 1% DOX-loaded microspheres with and without salt are shown in FIG. 4. The spectra of the DOX-loaded microspheres show no characteristic differences than those of control formulations; this may be the result of the relatively low ratio of drug to polymer.

### Thermal Analysis

DSC was used to determine the thermal properties of the DOX, PLGA, and the DOX-loaded PLGA formulations (Table 1). Thermal analysis was used to evaluate the interaction of the drug and the polymer within the microspheres as well as to determine the physical state of the DOX in each formulation since both these characteristics could affect the drug release kinetics. Thermal analysis of the bulk PLGA revealed a glass transition (T_{G}) at 47.73° C. and no melt due to its amorphous nature while DOX revealed a melting temperature (T_{M}) of 169.05° C. due to its crystalline nature.

The reduction in the glass transition temperature could be caused by the molecular dispersion of doxycycline within the polymeric matrix, which may result in the drug exerting a plasticizing effect upon the PLGA. Furthermore, work by Izumikawa et al., studying polymer and drug crystallinity employing PGA microspheres loaded with progesterone using the solvent evaporation method, revealed that the fast removal of solvent during polymer precipitation causes a loss of crystallinity to yield amorphous spheres (Izumikawa et al. 1991). While PLGA is fully amorphous; however, a similar phenomenon could explain the characteristic decrease in the T_{G} seen in our formulations. During our solvent removal process, we believe the phase inversion of the polymer occurs upon addition of the W/O/O double emulsion into the excess of petroleum ether, which extracts the solvent from the polymer solution very rapidly.

DSC analysis of the doxycycline-loaded PLGA formulations revealed the absence of a first-order transition in the range of the doxycycline melting temperature, which suggests that there may be a molecular dispersion of the doxycycline within the microspheres as well as the possibility that the encapsulated doxycycline precipitate in an amorphous state (Mathiowitz et al. 1999; Freiberg et al. 2004). However, the decrease in glass transition suggests that the absence of the doxycycline melt is the result of the molecular dispersion of the drug within the polymeric matrix; if the drug became amorphous but phase separated, the glass transition of the polymer would not have been suppressed. To confirm this hypothesis, XRD was used to further probe the physical state of the doxycycline within the microspheres.

### X-ray Diffraction Analysis

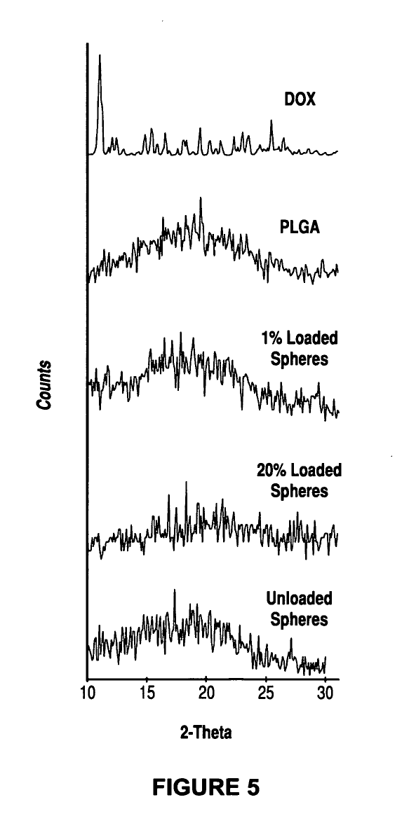

The crystallinity of unencapsulated DOX and DOX encapsulated within PLGA microspheres was evaluated using XRD. The XRD diffractograms for DOX, the raw polymer, DOX-loaded PLGA microspheres, and unloaded PLGA control microspheres is presented in FIG. 5. DOX displays several crystalline bands with the strongest at 11°, suggesting a crystalline nature. These peaks, indicative of crystallinity, were not depicted in the DOX-loaded PLGA microspheres for both low loading (1%) and high loading (20%), the latter which was prepared and analyzed only to confirm that the absence of diffraction at the lower loading was in fact due to the amorphous nature of the drug, after encapsulation, and not due to low detection levels in the sample.

In agreement with the results obtained via thermal analysis of the DOX-loaded microspheres, the lack of crystallinity of DOX in the diffractograms indicates dispersion of DOX in the amorphous state within the PLGA matrix. Since the drug is entrapped while dissolved in an aqueous phase followed by removal of water by sublimation during lyophilization, the encapsulated drug may not be able to re-crystallize during microsphere preparation. This is consistent with previous work by Yuksel et al in which XRD and DSC were used to investigate the crystallinity and drug-polymer interactions in acrylic microspheres containing nicardipine, a hydrophilic small molecule dihydropyridine calcium antagonist. Their work showed that although a physical mixture of drug and polymer exhibits crystallinity, the drug became amorphous following encapsulation in the microspheres (Yuksel et al. 1996).

Combining the results of thermal analysis by DSC with XRD analysis, we can conclude that there is a molecular dispersion of the doxycycline in the amorphous state, exerting a plasticizing effect on the PLGA matrix of the micro- and nanospheres as a result of the processing parameters used in our modified W/O/O double-emulsion solvent removal method.

### CONCLUSION

A modified water-in-oil-in-oil (W/O/O) double emulsion solvent removal method has been developed for the encapsulation of hydrophilic small molecule drugs in PLGA micro- and nanospheres. These studies with doxycycline have revealed that the manufacturing conditions result in higher encapsulation efficiencies than previous described in literature, likely due to the processing of the spheres in hydrophobic conditions that result in the rapid precipitation of the polymer into micro- and nanospheres. By incorporating high shear rates, the particle sizes of the spheres produced by this method have been significantly reduced to yield small diameter micro- and nanospheres as confirmed by Coulter laser diffraction particle size analysis, dynamic light scattering and scanning electron microscopy; this data suggests that the size of the polymer droplets in the continuous phase prior to solvent removal dictate final particle size. Most importantly, the encapsulation of doxycycline in PLGA using the solvent removal method resulted in the sustained release of the drug up to 85 days. This release followed the characteristic pattern of drugs from PLGA, with an initial release of drug trapped on the surface followed by the later, more sustained release of drug entrapped within the polymeric matrix. The ability to sustain release of a hydrophilic small molecule for up to 12 weeks is a significant improvement over previous attempts in the literature. Characterization of the spheres by differential scanning calorimetry and x-ray diffractometry revealed that the micro- and nanospheres contained a molecular dispersion of the doxycycline in the amorphous state, suggesting entrapment within the polymer as evidenced by the plasticizing effect of the drug on the polymer causing a reduction in glass transition temperature and the absence of characteristic peaks associated with crystalline doxycycline. This complete incorporation of the drug in the polymeric matrix suggests that the sustained release of doxycycline after the initial release is dependent on the degradation of the polymeric matrix rather than diffusive mechanisms. These results indicate that this modified W/O/O double emulsion solvent removal method is an attractive means of encapsulating hydrophilic small molecule drugs in PLGA micro- and nanospheres with high encapsulation efficiencies and sustained release kinetics for both therapeutic and tissue engineering applications.

TABLE 1
Encapsulation Efficiency and Mean Particle Size of Various Formulations

| | | | % | Particle Size | | Thermal Analysis | |
|---|---|---|---|---|---|---|---|
| | % DOX | % NaCl | Encapsulation | Volume | Number | T_{G} | T_{M} |
| Formulation | Loaded | Loaded | Efficiency | Average (μm) | Average (nm) | (° C.) | (° C.) |
| A | 10 | 0 | 51.1 ± 4.99 | 18.69 ± 13.48 | 774 ± 669 | 35.72 | — |
| B | 10 | 1 | 38.9 ± 5.44 | 13.47 ± 10.82 | 846 ± 756 | 34.72 | — |
| C | 5 | 0 | 70.1 ± 3.25 | 16.46 ± 12.46 | 770 ± 653 | 35.59 | — |
| D | 5 | 1 | 73.9 ± 3.74 | 12.92 ± 10.08 | 682 ± 570 | 36.47 | — |
| E | 1 | 0 | 69.6 ± 1.05 | 14.56 ± 11.79 | 796 ± 623 | 38.67 | — |
| F | 1 | 1 | 73.9 ± 9.22 | 12.42 ± 10.09 | 812 ± 656 | 37.06 | — |
| G | 0 | 0 | — | 16.75 ± 12.99 | 768 ± 635 | 37.75 | — |
| H | 0 | 1 | — | 14.24 ± 12.09 | 676 ± 532 | 38.07 | — |

Bulk DOX Tm: 169.05° C.; Bulk PLGA Tg: 47.73° C.

While this invention has been described as having preferred sequences, ranges, steps, materials, structures, components, features, and/or designs, it is understood that it is capable of further modifications, uses, and/or adaptations of the invention following in general the principle of the invention, and including such departures from the present disclosure as those come within the known or customary practice in the art to which the invention pertains, and as may be applied to the central features herein before setforth, and fall within the scope of the invention and of the limits of the appended claims.

### REFERENCES

The following references, and those cited in the disclosure herein, are hereby incorporated herein in their entirety by reference.
1. Ciombor, D. M. et al. 2006. Encapsulation of BSA using a modified W/O/O emulsion solvent removal method. Journal of Microencapsulation 23(2): 183-194.
2. Dahl, E. L. et al. 2006. Tetracyclines specifically target the apicoplast of the malaria parasite Plasmodium falciparum. Antimicrob Agents Chemother 50(9): 3124-3131.
3. Feng, K. et al. 2010. Novel antibacterial nanofibrous PLLA scaffolds. J Control Release 146(3): 363-369.
4. Freiberg, S., & Zhu, X. X. 2004. Polymer microspheres for controlled drug release. Int J Pharm 282(1-2): 1-18.
5. Izumikawa, S., Yoshioka, S., Aso, Y., & Takeda, Y. 1991. Preparation of poly(l-lactide) microspheres of different crystalline morphology and effect of crystalline morphology on drug release rate. Journal of Controlled Release 15(2): 133-140.
6. Lalloo, D. G. et al. 2007. UK malaria treatment guidelines. J Infect 54(2): 111-121.
7. Liu, D. Z. et al. 2004. Effects of alginate coated on PLGA microspheres for delivery tetracycline hydrochloride to periodontal pockets. Journal of Microencapsulation 21(6): 643-652.
8. Maatta, M. et al. 2006. Tear fluid levels of MMP-8 are elevated in ocular rosacea—treatment effect of oral doxycycline. Graefes Arch Clin Exp Ophthalmol 244(8): 957-962.
9. Mathiowitz, E., Saltzman, W. M., Domb, A., Dor, P., & Langer, R. 1988. Polyanhydride microspheres as drug carriers. II. Microencapsulation by solvent removal. J Appl Polym Sci 35(3): 755-774.
10. Mathiowitz, E., Kline, D., & Langer, R. 1990. Morphology of polyanhydride microsphere delivery systems. Scanning microscopy 4(2): 329-340.
11. Mathiowitz, E., Kretiz, M., & Brannon-Peppas, L. 1999. Microencapsulation 1st ed. New York, N.Y.: John Wiley & Sons, Inc.
12. Mundargi, R. C. et al. 2007. Development and evaluation of novel biodegradable microspheres based on poly(d,l-lactide-co-glycolide) and poly(epsilon-caprolactone) for controlled delivery of doxycycline in the treatment of human periodontal pocket: in vitro and in vivo studies. J Control Release 119(1): 59-68.
13. Patel, P. et al. 2008. Microencapsulation of doxycycline into poly (lactide-co-glycolide) by spray drying technique: Effect of polymer molecular weight on process parameters. Journal of Applied Polymer Science 108: 4038-4046.
14. Quarterman, M. J. et al. 1997. Ocular rosacea. Signs, symptoms, and tear studies before and after treatment with doxycycline. Arch Dermatol 133(1): 49-54.
15. Smith, K., & Leyden, J. J. 2005. Safety of doxycycline and minocycline: a systematic review. Clin Ther 27(9): 1329-1342.
16. Stewart, M. W., Liesegang, T. J., & Schwam, B. L. 2005. Chlamydia conjunctivitis and central retinal vein occlusion. Am J Ophthalmol 140(1): 161-162.
17. Tan, L. P., Hidayat, A., Lao, L. L., & Quah, L. F. 2009. Release of hydrophilic drug from biodegradable polymer blends. J Biomater Sci Polym Ed 20(10): 1381-1392.
18. Webber, W. L., Lago, F., Thanos, C., & Mathiowitz, E.
1998. Characterization of soluble, salt-loaded, degradable PLGA films and their release of tetracycline. Journal of biomedical materials research 41(1): 18-29.
19. Yuksel, N., Tincer, T., & Baykara, T. 1996. Interaction between nicardipine hydrochloride and polymeric microspheres for a controlled release system. International journal of pharmaceutics 140(2): 145-154.
20. Zeidner, N. S. et al. 2004. Sustained-release formulation of doxycycline hyclate for prophylaxis of tick bite infection in a murine model of Lyme borreliosis. Antimicrob Agents Chemother 48(7): 2697-2699.
21. Zhang, Y., Zale, S., Sawyer, L., & Bernstein, H. 1997. Effects of metal salts on poly(DL-lactide-co-glycolide) polymer hydrolysis. Journal of biomedical materials research 34(4): 531-538.

What is claimed is:

1. A method of encapsulating a material, comprising the steps of:
a) providing a polymer solution including a solvent, and an aqueous solution including a hydrophilic material;
b) mixing the polymer and aqueous solutions;
c) sonicating the solution obtained in step b) to obtain a water-in-oil emulsion;
d) mixing the water-in-oil emulsion with an oil solution;
e) sonicating the solution obtained in step d) to obtain a water-in-oil-in-oil emulsion; and
f) stirring the water-in-oil-in-oil emulsion in a bath to form a precipitate comprising an encapsulated material, and separate the solvent.

2. The method of claim 1, wherein:
the step c) comprises sonicating the solution obtained in step b) in an ultrasonic homogenizer at about 40% amplitude for about one minute.

3. The method of claim 2, wherein:
the step e) comprises sonicating the solution obtained in step d) for about two minutes.

4. The method of claim 3, wherein:
the step f) comprises stirring the water-in-oil-in-oil emulsion in a bath at about 2000 rpm for about five minutes.

5. The method of claim 1, wherein:
the hydrophilic material comprises a low molecular weight compound or molecule.

6. The method of claim 1, wherein:
the hydrophilic material comprises an antibiotic.

7. The method of claim 6, wherein:
the antibiotic comprises a tetracycline or a derivative thereof.

8. The method of claim 6, wherein:
the antibiotic comprises doxycycline (DOX).

9. The method of claim 7, wherein:
the polymer solution comprises poly(lactic-co-glycolic acid) (PLGA).

10. The method of claim 1, wherein:
the oil solution comprises a solution of an organic solvent and silicon oil.

11. The method of claim 10, wherein:
the organic solvent comprises dichloromethane (DCM).

12. The method of claim 1, wherein:
the bath comprises petroleum ether.

13. The method of claim 5, wherein:
the encapsulated material comprises microspheres loaded with the low molecular weight compound or molecule.

14. The method of claim 13, wherein:
the compound or molecule comprises an antibiotic.

15. The method of claim 14, wherein:
the antibiotic comprises doxycycline (DOX).

16. The method of claim 13, wherein:
a plurality of microspheres are loaded with upto 20% by weight of the compound or molecule.

17. The method of claim 13, wherein:
the microspheres have an average diameter of from about 600 nm to about 19 μm.

18. The method of claim 13, wherein:
a plurality of microspheres have a capacity of upto 85 days for release of the compound or molecule.

19. The method of claim 13, wherein:
a plurality of spheres have a glass transition (T_{G}) temperature of from about 34° to about 39°.

20. A sustained release product made in accordance with the method of claim 1.
